(19) United States
(12) Patent Application Publication
(10) Pub. No.: US 2026/0245654 A1
(43) Pub. Date: Aug. 20, 2026

(54) METHOD AND DEVICE FOR ESTIMATING BINDING BETWEEN PEPTIDE AND T CELL RECEPTOR

(71) Applicant: GENINUS INC., Songpa-gu, Seoul (KR)

(72) Inventors: Kyu Young SONG, Gangnam-gu, Seoul (KR); Jong Myeon Park, Songpa-gu, Seoul (KR); Young Ae Park, Songpa-gu, Seoul (KR); Jong Hoo Lee, Songpa-gu, Seoul (KR)

(21) Appl. No.: 19/485,123

(22) PCT Filed: Jun. 22, 2023
(86) PCT No.: PCT/KR2023/008703

§ 371 Date: Nov. 17, 2025

(30) Foreign Application Priority Data
Jun. 24, 2022 (KR) 10-2022-0077714
Jun. 21, 2023 (KR) 10-2023-0079811

(51) Int. Cl. G16B 15/30; G16B 40/20

(52) U.S. Cl. CPC G16B 15/30; G16B 40/20

(57) ABSTRACT

Examples of the present specification are directed to techniques for estimating binding between a peptide and a TCR.

A method for generating a learning model for estimating peptide-T cell receptor (TCR) binding, according to one embodiment of the present specification for achieving the above-described objective, may comprise: generating a plurality of major histocompatibility complex-T cell receptor (MHC-TCR) structures by using an amino acid sequence as an input to a first learning model; obtaining a plurality of first pMHC-TCR structures; comparing the plurality of first pMHC-TCR structures and the plurality of MHC-TCR structures and identifying a plurality of peptides corresponding to the plurality of first pMHC-TCR structures; generating a second pMHC-TCR structure for each of the plurality of peptides, on the basis of the plurality of MHC-TCR structures; calculating structural energy of the second pMHC-TCR structure; generating a data set on the basis of the structural energy and the plurality of peptides; and generating a second learning model that estimates whether the peptide will bind to the TCR on the basis of the data set.

### TECHNICAL FIELD

The present disclosure relates to techniques for estimating binding between a peptide and a TCR.

### BACKGROUND ART

Cancer is a disease in which certain cells of the body uncontrollably grow and spread to other parts of the body. Cancer is characterized by these abnormal cells exhibiting uncontrolled growth and metastasis and is a disease with high death rates negatively affecting all organs and tissues of the body and often leading to death.

In such tumor cells, neoantigens, which are antigenic substances that activate immune responses in the human body, are produced. The neoantigens, as antigenic peptides specifically expressed only in cancer cells, are presented by the major histocompatibility complex (MHC) and bound to a T cell receptor (TCR) to induce an immune response.

Vaccination therapy using neoantigens may induce tumor-specific T cell responses, because vaccination targets neoantigens not expressed in normal cells but expressed only in cancer cells. In addition, vaccination therapy is free from adverse effects and enables precision treatment via T-cell therapy based on individual genetic mutations. Therapeutic vaccines based on such tumor-specific neoantigens are anticipated as next-generation personalized immunotherapies for cancer.

In previous studies, research has been conducted to predict neoantigen substances presented by the MHC in individual patients based on amino acid sequences of the MHC and gene expression levels. However, when neoantigens presented by the MHC are identified in tumor cells by using the predicted candidate neoantigens, only about one-third actually elicited TCR responses. The reason why only about one-third of the neoantigen candidates exhibit TCR responses is that the administered candidates are not presented by the MHC, or if presented, fail to bind to TCRs.

In cellular immune responses, T cells serve as key mediators of adaptive immunity by recognizing peptide epitopes presented on MHC molecules through their T cell receptors (TCRs). However, because the immune repertoire comprises a vast diversity of T cells with distinct peptide-MHC specificities, identifying the precise peptide-MHC-TCR complex structures formed in each patient remains highly costly and time-consuming.

The above-described background is technical information that the inventors possessed for deriving the present disclosure, or that the inventors acquired during the course of deriving the present disclosure, and therefore cannot necessarily be regarded as prior art publicly available before the filing of the present disclosure.

### DISCLOSURE OF INVENTION

### Technical Problem

The present disclosure provides techniques for estimating binding between a peptide and a T cell receptor (TCR) to address the problems described above.

### Solution to Problem

A method for generating a learning model for estimating peptide-T cell receptor (TCR) binding according to an aspect of the present disclosure for achieving the above-described objective may include: generating a plurality of major histocompatibility complex-T cell receptor (MHC-TCR) structures by using an amino acid sequence as an input to a first learning model; obtaining a plurality of first pMHC-TCR structures; comparing the plurality of first pMHC-TCR structures and the plurality of MHC-TCR structures and identifying a plurality of peptides corresponding to the plurality of first pMHC-TCR structures; generating a second pMHC-TCR structure for each of the plurality of peptides, on the basis of the plurality of MHC-TCR structures; calculating structural energy of the second pMHC-TCR structure; generating a data set on the basis of the structural energy and the plurality of peptides; and generating a second learning model configured to estimate whether a peptide will bind to a TCR, on the basis of the data set.

The identifying of the plurality of peptides may include matching one first pMHC-TCR structure of the plurality of first pMHC-TCR structures with one MHC-TCR structure of the plurality of MHC-TCR structures, based on structural similarity

The identifying of the plurality of peptides may include determining a peptide bound to the first pMHC-TCR as a first peptide that binds to a TCR, when MHC-TCR sequences are identical in the matched structure of the first pMHC-TCR structure and the MHC-TCR structure.

The identifying of the plurality of peptides may include determining a peptide bound to the first pMHC-TCR as a second peptide that does not bind to a TCR, when MHC-TCR sequences are identical in the matched structure of the first pMHC-TCR structure and the MHC-TCR structure.

The identifying of the plurality of peptides may include determining a peptide bound to the first pMHC-TCR as a third peptide that binds to the MHC but does not bind to a TCR, when the peptide bound to the first pMHC-TCR is estimated to bind to the MHC of the MHC-TCR structure in the matched structure of the first pMHC-TCR structure and the MHC-TCR structure.

The generating of the data set may include using structural energy as a feature and labeling the first peptide within a first value and the second peptide and the third peptide with a second value.

The generating of the second pMHC-TCR structure may include: generating a pMHC structure for each of the plurality of peptides, on the basis of a plurality of MHC structures respectively included in the plurality of MHC-TCR structures and STRUMP-I; matching the pMHC structure with one of the plurality of MHC-TCR structures on the basis of a similar MHC; and removing the MHC structure corresponding to the pMHC structure, and generating a pMHC-TCR structure on the basis of the MHC-TCR structure matched with a peptide corresponding to the pMHC structure.

The calculating of the structural energy may include: performing structural optimization of a backbone and a side chain of the second pMHC-TCR structure; and calculating energy between the pMHC and the TCR, energy between the MHC and the TCR, and energy between the peptide and the TCR in the second pMHC-TCR structure.

The obtaining of the plurality of first pMHC-TCR structures may include obtaining the first pMHC-TCR structures from an RCSB_PDB database.

The amino acid sequence may include sequences of MHC, TCR-Alpha, and TCR-Beta connected by a linker formed of glutamic acid.

A method for estimating peptide-T cell receptor (TCR) binding according to an aspect of the present disclosure for achieving the above-described objective may include may include: obtaining MHC-TCR structures and amino acid sequences of a plurality of peptides; generating a pMHC-TCR structure for each of the plurality of peptides, on the basis of the MHC-TCR structures; calculating structural energy of the pMHC-TCR structure; and estimating whether at least one of the plurality of peptides will bind to the TCR by using the structural energy as an input to a second learning model configured to estimate whether the peptide will bind to the TCR.

A computer device for generating a learning model for estimating peptide-T cell receptor (TCR) binding according to an aspect of the present disclosure for achieving the above-described objective may include: a memory including instructions; and a processor configured to execute the instructions, wherein the processor executes instructions for generating a plurality of major histocompatibility complex-T cell receptor (MHC-TCR) structures by using an amino acid sequence as an input to a first learning model; obtaining a plurality of first pMHC-TCR structures; comparing the plurality of first pMHC-TCR structures and the plurality of MHC-TCR structures and identifying a plurality of peptides corresponding to the plurality of first pMHC-TCR structures; generating a second pMHC-TCR structure for each of the plurality of peptides, on the basis of the plurality of MHC-TCR structures; calculating structural energy of the second pMHC-TCR structure; generating a data set on the basis of the structural energy and the plurality of peptides; and generating a second learning model configured to estimate whether a peptide will bind to a TCR, on the basis of the data set.

A computer device for estimating peptide-T cell receptor (TCR) binding according to an aspect of the present disclosure for achieving the above-described objective may include: a memory including instructions; and a processor configured to execute the instructions, wherein the processor executes instructions for obtaining MHC-TCR structures and amino acid sequences of a plurality of peptides; generating a pMHC-TCR structure for each of the plurality of peptides, on the basis of the MHC-TCR structures; calculating structural energy of the pMHC-TCR structure; and estimating whether at least one of the plurality of peptides will bind to a TCR by using the structural energy as an input to a second learning model configured to estimate whether a peptide will bind to a TCR.

### Advantageous Effects of Invention

According to an embodiment of the present disclosure, peptide-TCR binding may be predicted more effectively.

According to an embodiment of the present disclosure, reduction of time and cost in personalized anticancer therapy may be maximized by conducting the analysis in an in silico environment.

The effects achieved are not limited to those mentioned above, and any other effects not mentioned herein will be understood by those skilled in the art to which the present disclosure belongs.

### BRIEF DESCRIPTION OF DRAWINGS

FIG. 1 is a view schematically illustrating a peptide-major histocompatibility complex-T cell receptor (pMHC-TCR) protein structure according to an embodiment of the present disclosure.

FIG. 2 is a flowchart illustrating an operation of estimating peptide-T cell receptor (TCR) binding, performed by a computer device according to an embodiment of the present disclosure.

FIG. 3 is a flowchart illustrating an operation of learning a learning model that estimates peptide-TCR binding, performed by a computer device according to an embodiment of the present disclosure.

FIG. 4 is a view schematically illustrating a major histocompatibility complex-T cell receptor (MHC-TCR) protein structure according to an embodiment of the present disclosure.

FIG. 5 is a view for describing an operation of identifying peptides for generating a data set, performed by a computer device according to an embodiment of the present disclosure.

FIG. 6 is a view for describing an operation of generating a pMHC-TCR protein structure according to an embodiment of the present disclosure.

FIG. 7 is a view illustrating an effect of a method of estimating binding, according to an embodiment of the present disclosure.

FIG. 8 is a block diagram schematically illustrating the configuration of a computer device according to an embodiment of the present disclosure.

### MODE FOR THE INVENTION

As the present disclosure below allows for various changes and numerous embodiments, particular embodiments will be illustrated in the drawings and described in detail in the written description. Effects and features of the present disclosure and a method of achieving the effects and features will be apparent by referring to embodiments described below in connection with the accompanying drawings. However, the present disclosure is not restricted by these embodiments but can be implemented in many different forms.

Each block may represent a module, a segment, or a portion of code including one or more executable instructions for performing specific logical functions. In another embodiment, it should be noted that the functions described for each block may be executed in an order different from that set forth herein. For example, although two blocks are illustrated sequentially, the functions associated with the respective blocks may instead be performed substantially concurrently, or in reverse order, depending on execution conditions or the operating environment. An expression used in the singular encompasses the expression of the plural unless it has a clearly different meaning in the context.

In the following embodiments, it is to be understood that the terms “include” or “have” are intended to indicate the existence of elements disclosed in the specification and are not intended to preclude the possibility that one or more other elements may exist or may be added.

Instructions executed by a processor of a computer or other programmable data processing apparatus may constitute means for performing the functions described with reference to a flowchart or block diagram. Instructions may be loaded onto a computer or other device, thereby generating processes that are executed to carry out a series of operations.

In this regard, the term ‘-unit’ refers to a component that performs a specific function, which may be implemented by software, or by hardware such as a field programmable gate array (FPGA) or an application specific integrated circuit (ASIC). However, the ‘-unit’ is not limited to being performed by software or hardware. The ‘-unit’ may also exist in the form of data stored on an addressable storage medium, and may be configured to allow one or more processors execute specific functions.

The software may include a computer program, code, instruction, or a combination of one or more thereof, and may configure a processing device to operate as desired, or may direct the processing device independently or collectively. The software and/or data may be embodied, either permanently or temporarily, in any type of machine, component, physical device, virtual equipment, computer storage medium or device, or transmitted signal wave, to be interpreted by a processing device or to provide instructions or data to the processing device. Software may be distributed over computer systems connected by a network and may be stored or executed in a distributed fashion. The software and the data may be stored in one or more computer-readable recording media.

A learning model according to the present disclosure is a model trained to recognize specific patterns, providing algorithms that enable training on a dataset as well as inference and further learning from the data. Once trained, the model may be applied to previously unseen data for inference and prediction. Alternatively, the learning model is an example of an artificial neural network model that mimics brain neurons and is not necessarily limited to a specific algorithm.

The terms used herein are merely used to describe particular embodiments and are not intended to limit the present disclosure. An expression used in the singular encompasses the expression of the plural, unless it has a clearly different meaning in the context. In the present specification, it is to be understood that the terms such as “including” or “having,” etc., are intended to indicate the existence of the features, numbers, operations, elements, parts, or combinations thereof disclosed in the specification, and are not intended to preclude the possibility that one or more other features, numbers, operations, elements, parts, or combinations thereof may exist or may be added. It will be understood that, although the terms first, second, etc. may be used herein to describe various elements, these elements should not be limited by these terms. These terms are only used to distinguish one element from another.

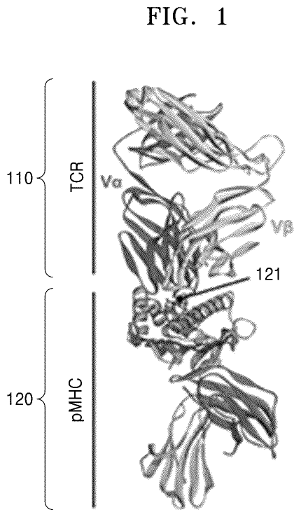

FIG. 1 is a view schematically illustrating a peptide-major histocompatibility complex-T cell receptor (pMHC-TCR) protein structure according to an embodiment of the present disclosure.

Referring to FIG. 1, the pMHC-TCR may include a T cell receptor (TCR) 110, a peptide-major histocompatibility complex (pMHC) 120, and a peptide 121.

The TCR 110 refers to a T cell receptor and is a major protein structure that initiates an immune response to a specific antigen. This TCR reacts with a specific antigen, particularly, in this case, with a specific antigen expressed by the peptide 121.

The pMHC 120 is a major histocompatibility complex (MHC) bound to a peptide. Specifically, the MHC is located on the surface of cells and may deliver a peptide generated in the cells to the surface. Such a peptide is recognized by a TCR, and thus T cells may recognize foreign substances or pathogens existing within the cells.

The peptide 121 is a small protein fragment presented by the MHC. A certain peptide is recognized by a certain TCR, inducing T-cell activation. This peptide-MHC-TCR complex plays important roles in the immune response, and according to the present disclosure, actual binding between the peptide and the TCR may be predicted based on structure and energy of the complex.

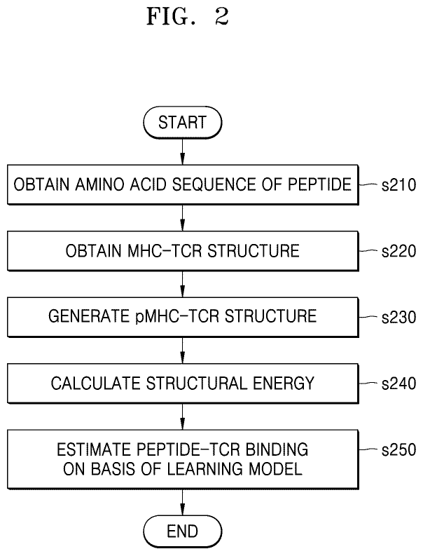

FIG. 2 is a flowchart illustrating an operation of estimating peptide-T cell receptor (TCR) binding, performing by a computer device according to an embodiment of the present disclosure.

Referring to FIG. 2, the computer device may obtain an amino acid sequence of a plurality of peptides in S210. For example, the plurality of peptides may constitute a pool of neoantigen candidates corresponding to cancer cells of a patient.

The computer device according to an embodiment may obtain an MHC-TCR structure in S220. Specifically, the computer device may identify an amino acid sequence of the TCR from T cells of the patient by sequence analysis and identify an amino acid sequence of the MHC corresponding to normal cells of the patient. In this case, the MHC corresponding to the normal cells of the patient may correspond to a human leukocyte antigen (HLA). The computer device may obtain an MHC-TCR structure by using the amino acid sequence of the TCR and the amino acid sequence of the MHC.

In S230, the computer device according to an embodiment may generate a pMHC-TCR structure based on the MHC-TCR structures for each of the plurality of peptides. The computer device may determine a p-MHC pair by estimating a p-MHC binding strength and generate and optimize the pMHC-TCR structure by using the corresponding MHC-TCR structure. For example, the computer device may perform structural optimization on the pMHC-TCR protein complex by using the MINIMIZE tool of Tinker. This optimization process may include a backbone and a side chain of the protein structure.

The computer device according to an embodiment may calculate structural energy of the pMHC-TCR structure in S240. For example, the computer device may calculate energy parameter values related to pMHC-TCR. The computer device calculates energy parameter values generated from the pMHC-TCR protein structure by using the ANALYZE tool of Tinker.

TABLE 1
Tinker energy parameter

Total Potential Energy
Hydrophobic_PMF
Electrostatic
Bond_Stretching-kcal
Bond_Stretching-inter
Angle_Bending-kcal
Angle_Bending-inter
Improper_Torsion-kcal
Improper_Torsion-inter
Torsional_Angle-kcal
Torsional_Angle-inter
Van_der_Waals-kcal
Van_der_Waals-inter
Charge-Charge-kcal
Charge-Charge-inter
bonded_energy-kcal
non_bonded_energy-kcal
bonded_energy-inter
non_bonded_energy-inter
solvation_energy

Table 1 is a list of energy parameters obtained by the analysis of Tinker.

In addition, the computer device may calculate energy parameter values generated from each chain of the protein structure based on the structurally optimized pMHC-TCR structure by using Foldx's AnalyzeComplex. Specifically, the computer device calculates chain-specific energy parameter values for a total of three combinations of pMHC and TCR, MHC and TCR, and peptide and TCR for chain-specific energy parameter values.

TABLE 2
foldx energy parameter

| | |
|---|---|
| StabilityGroup1 | water bridge |
| StabilityGroup2 | disulfide |
| IntraclashesGroup1 | electrostatic kon |
| IntraclashesGroup2 | partial covalent bonds |
| Interaction Energy | energy Ionisation |
| Backbone Hbond | Entropy Complex |
| Sidechain Hbond | Number of Residues |
| Van der Waals | Interface Residues |
| Electrostatics | Interface Residues Clashing |
| Solvation Polar | Interface Residues VdW Clashing |
| Solvation Hydrophobic | Interface Residues BB Clashing |
| Van der Waals clashes | |
| entropy sidechain | |
| entropy mainchain | |
| sloop_entropy | |
| mloop_entropy | |
| cis_bond | |
| torsional clash | |
| backbone clash | |
| helix dipole | |

Table 2 shows a list of energy parameters obtained by Foldx's AnalyzeComplex.

In S250, the computer device according to an embodiment may estimate whether at least one of the plurality of peptides will bind to the TCR by using the structural energy as an input to a learning model that estimates whether the peptide will bind to the TCR. For example, the computer device may estimate whether at least one of the plurality of peptides will bind to the TCR by inputting energy parameter values and chain-specific energy parameter values related to pMHC-TCR to a learning model generated by using energy parameters and chain-specific energy parameters related to pMHC-TCR as features and the binding status as a label. For example, the computer device may estimate whether the peptide will bind to the TCR by calculating the structural energy by including at least one of the parameters listed in the parameter lists of Tables 1 and 2, and inputting the structural energy to the learning model.

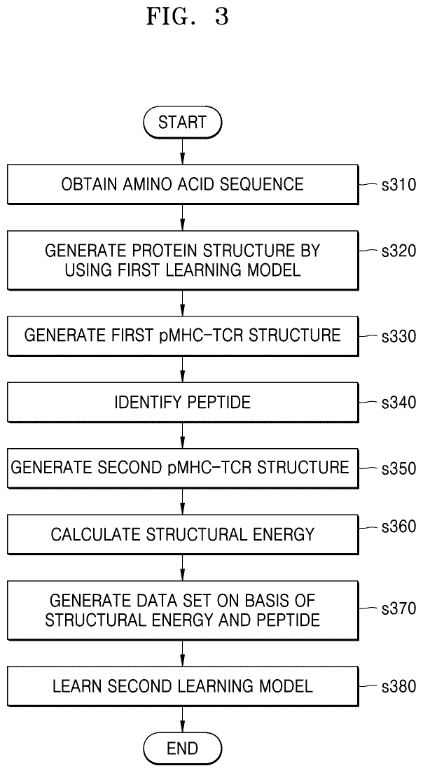

The learning model will be described below in more detail with reference to FIG. 3.

FIG. 3 is a flowchart illustrating an operation of a learning model that estimates peptide-TCR binding, performed by a computer device according to an embodiment of the present disclosure.

Referring to FIG. 3, in S310, the computer device may obtain an amino acid sequence. For example, the computer device may connect amino acid sequences of MHC, TCR-Alpha, and TCR-Beta by amino acid sequences consisting of glutamic acid, called as a linker, to construct a single protein structure.

Specifically, the computer device may obtain an amino acid sequence by inserting 20 glutamic acid sequences between the MHC and the TCR-Alpha, and inserting 15 glutamic acid sequences between the TCR-Alpha and the TCR-Beta.

For example, the amino acid sequences may be obtained as shown in Table 3 below.

TABLE 3

| Protien | |
|---|---|
| structure | Amino acid saquencas |
| MHC | GSHSMRYFFTSVSRPGRGEPRFIAVGYVDD |
| | TQFVRFDSDAASQRMEPRAPWIEQEGPEYW |
| | DGETRKVKAHSQTHRVDLGTLRGYYNQSEA |
| | GSHTVQRMYGCDVGSDWRFLRGYHQYAYDG |
| | KDYIALKEDLRSWTAADMAAQTTKHKWEAA |
| | HVAEQLRAYLEGTCVEWLRRYLENGKETLQ |
| | RT |
| TCR(Alpha) | KEVEQNSGPLSVPEGAIASLNCTYSDRGSQ |
| | SFFWYRQYSGKSPELIMSIVSNGDKEDGRF |
| | TAQLNKASQYVSLLIRDSQPSDSATYLCAV |
| | TTDSWGKLQFGAGTQVVVTP |
| TCR(Beta) | NAGVTQTPKFQVLKTGQSMTLQCAQDMNHE |
| | YMSWVRQDPGMGLRLIHYSVGAGITDQGEV |
| | PNGYNVSRSTTEDFPLRLLSAAPSQTSVVF |
| | CASRPGLAGGRPEQYFGPGTRLTVT |
| MHC + | GSHSMRYFFTSVSRPGRGEPRFIAVGYVDD |
| G*20 + | TQFVRFDSDAASQRMEPRAPWIEQEGPEYW |
| TCR(Alpha) + | DGETRKVKAHSQTHRVDLGTLRGYYNQSEA |
| G*15 + | GSHTVQRMYGCDVGSDWRFLRGYHQYAYDG |
| TCR(Beta) | KDYIALKEDLRSWTAADMAAQTTKHKWEAA |
| | HVAEQLRAYLEGTCVEWLRRYLENGKETLQ |
| | RTGGGGGGGGGGGGGGGGGGGGKEVEQNSG |
| | PLSVPEGAIASLNCTYSDRGSQSFFWYRQY |
| | SGKSPELIMSIVSNGDKEDGRFTAQLNKAS |
| | QYVSLLIRDSQPSDSATYLCAVTTDSWGKL |
| | QFGAGTQVVVTPGGGGGGGGGGGGGGGNAG |
| | VTQTPKFQVLKTGQSMTLQCAQDMNHEYMS |
| | WVRQDPGMGLRLIHYSVGAGITDQGEVPNG |
| | YNVSRSTTEDFPLRLLSAAPSQTSVVFCAS |
| | RPGLAGGRPEQYFGPGTRLTVT |

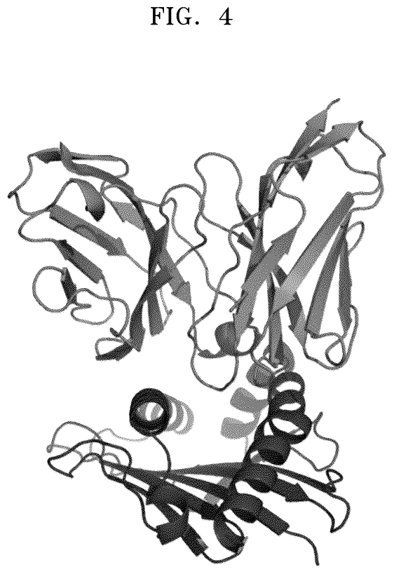

In S320, the computer device according to an embodiment may generate a plurality of major histocompatibility complex (MHC) structures and a major histocompatibility complex-T cell receptor (MHC-TCR) structures using the amino acid sequence as an input to the first learning model. For example, the computer device may generate an MHC protein structure and an MHC-TCR protein structure in the form consisting of MHC and TCR without a peptide by using the sequence data of MHC (sequence)+G*20 (sequence)+TCR-Alpha (sequence)+G*15 (sequence)+TCR-Beta (sequence) in Table 1 as an input to Alphafold. The computer device may remove the linker (20 glutamic acid residues and 15 glutamic acid residues) used in the formation of the structure after generating the MHC-TCR structure. FIG. 4 schematically illustrates the MHC-TCR protein structure generated by using the amino acid sequence as an input to the first learning model, performed by the computer device according to an embodiment of the present disclosure.

In S330, the computer device according to an embodiment may obtain a plurality of first pMHC-TCR structures. The plurality of first pMHC-TCR structures may be obtained from pMHC-TCR structure data known in the art. For example, the computer device may obtain publicly known first pMHC-TCR structures by using RCSB_PDB database including information on protein structures obtained by X-ray crystallography.

In S340, the computer device according to an embodiment may select an optimized structure from the plurality of MHC-TCR structures by comparing the plurality of first pMHC-TCR structures and the plurality of MHC-TCR structures and identifying a plurality of peptides corresponding to the plurality of first pMHC-TCR structures.

The computer device may match one first pMHC-TCR structure of the plurality of first pMHC-TCR structures with one MHC-TCR structure of the plurality of MHC-TCR structures, on the basis of structural similarity. Specifically, the computer device may generate a plurality of results of protein structure prediction by using the first learning model. Among them, the most optimized structure may be selected and used for the matching. For example, the computer device may select a similar MHC-TCR structure model by comparing the MHC-TCR structure model generated by Alphafold with the first pMHC-TCR structure. Specifically, the computer device may select the MHC-TCR structure on the basis of the Fnat value and the CAPRI criteria. The Fnat value indicates a proportion of amino acids, which are properly bound, in a certain protein structure, and the CAPRI indicates standard evaluation criteria for protein-protein interaction prediction. The computer device may consider only models having an Fnat value of 0.4 or more. Specifically, the Fnat value is defined as the number of amino acids involved in binding in a binding site of the first pMHC-TCR structure, excluding the peptide, divided by the number of amino acids involved in binding in the MHC-TCR generated by AlphaFold. The computer device may further introduce additional criteria in the case where two or more MHC-TCR structures are selected based on the Fnat value and the CAPRI criteria. For example, the computer device may select a structure with a high Fnat value and a low i-RMSD value. The i-RMSD may indicate a dispersion value of positional difference relative to the first pMHC-TCR.

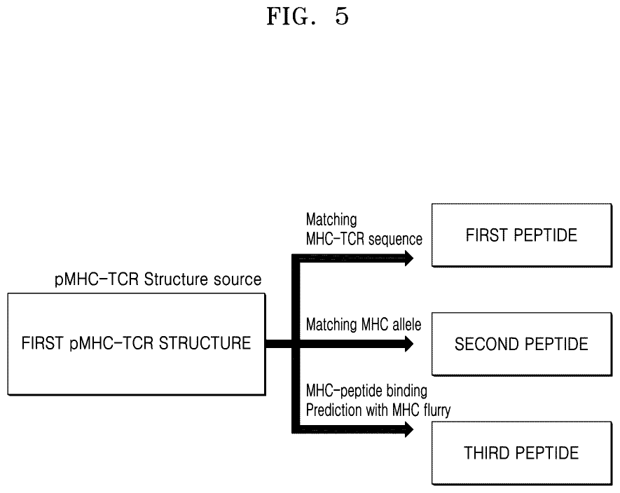

The computer device may identify the plurality of peptides corresponding to the plurality of first pMHC-TCR structures. As shown in FIG. 5, in the case where the sequences of the MHC-TCRs are the same in the matched structure of the first pMHC-TCR structure and the MHC-TCR structure, the computer device may determine a peptide to bind bound to the first pMHC-TCR as a first peptide that binds to the TCR.

When MHC-TCR sequences are identical in the matched structure of the first pMHC-TCR structure and the MHC-TCR structure, the computer device may determine a peptide bound to the first pMHC-TCR as a second peptide that does not bind to a TCR.

In addition, in the case where a peptide bound to the first pMHC-TCR is estimated to bind to the MHC of the MHC-TCR structure in the matched structure of the first pMHC-TCR structure and the MHC-TCR structure, the computer device may determine the peptide bound to the first pMHC-TCR as a third peptide that binds to the MHC but does not bind to the TCR. In this regard, the computer device may use MHC flurry (open-source package for MHC I binding prediction) to estimate that the peptide bound to the first pMHC-TCR will bind to the MHC of the MHC-TCR structure.

In S350, the computer device according to an embodiment may generate a second pMHC-TCR structure on the basis of the plurality of MHC-TCR structures for the plurality of peptides.

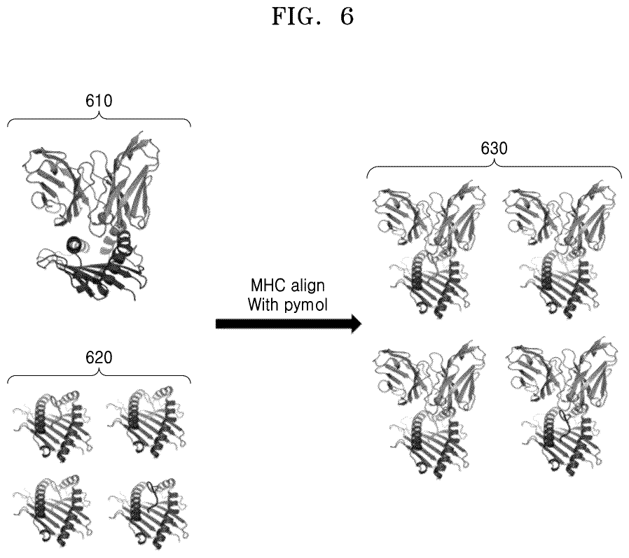

For example, the computer device may generate pMHC structures using STRUMP-I to predict structures of the protein-peptide complex, with respect to a plurality of MHC structures included in each of a plurality of MHC-TCR structures, for each of the plurality of peptides. Then, as shown in FIG. 6, the computer device may align the generated pMHC structure 620 and the MHC-TCR structure 610 previously generated by Alphafold, on the basis of MHC, by using PyMOL's superpose.

The computer device may generate a second pMHC-TCR structure 630 by removing the MHC from the aligned pMHC structure 620 and binding the residual peptide with the MHC-TCR structure 610. The computer device may also reassign each chain of the peptide-MHC-TCR (Alpha)-TCR (Beta) in the course of the process.

In S360, the computer device according to an embodiment may calculate structural energy of the second pMHC-TCR structure. This corresponds to the method of calculating the structural energy in S240 of FIG. 2. That is, the computer device may calculate energy parameter values and chain-specific energy parameter values related to pMHC-TCR.

In S370, the computer device according to an embodiment may generate a data set on the basis of the structural energy and the plurality of peptides. For example, the computer device may generate a data set by using the energy parameter and the chain-specific energy parameter related to the second pMHC-TCR as a feature and the peptide identified in S340 as a label.

The computer device may generate a data set by using a label configured to classify the first peptide as binding to the TCR and the second and third peptides as not binding to the TCR.

In S380, the computer device according to an embodiment may generate a second learning model that estimates whether the peptide will bind to the TCR on the basis of the data set. The second learning model corresponds to the learning model in FIG. 2, and may be a model using at least one of the algorithms, including XGBoost, Random Forest, Support Vector Machine (SVM), Neural Network, Logistic Regression, K-Nearest Neighbors (K-NN), and Naïve Bayes. The computer device may generate the second learning model by using the data set and at least one algorithm.

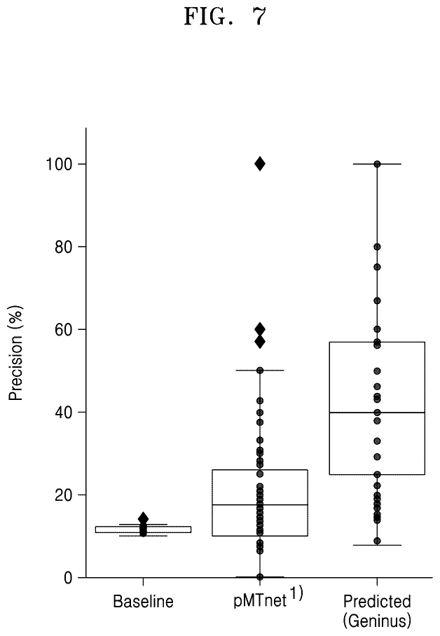

FIG. 7 is a view illustrating the effect of a method of estimating binding according to an embodiment of the present disclosure. Specifically, each point in FIG. 7 represents a result of performance evaluation obtained by splitting the fully labeled data set into a train set and a test set. A learning model (Geninus), which estimates whether a peptide will bind to the TCR by using XGBoost algorithm and uses foldx-Interaction Energy, foldx-Interface Residues, foldx-IntraclashesGroup1, foldx-Sidechain Hbond, foldx-Solvation Hydrophobic, foldx-Van der Waals clashes, foldx-entropy mainchain, foldx-entropy sidechain, tinker-Improper Torsion, tinker-Torsional Angle, and tinker-Intermolecular Energy as a feature and using the binding status as a label, was compared with pMHC-TCR binding prediction network (pMTnet) that is a peptide-TCR binding prediction model and a baseline obtained by random selectin from the test set in pMTnet.

As shown in FIG. 7, upon comparison with the pMTnet, about twofold improvement in performance is obtained.

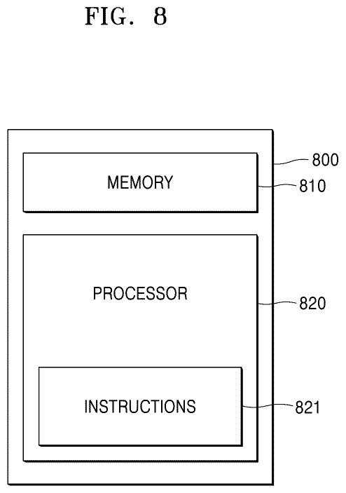

FIG. 8 is a block diagram schematically illustrating a configuration 800 of a computer device according to an embodiment of the present disclosure.

A memory 810, as a computer-readable recording medium, may include random access memory (RAM), read only memory (ROM), and a permanent mass storage device such as a disk drive. In addition, the memory 810 may store an operating system and at least one program code. Such software components may be loaded, via a drive mechanism, from a separate computer-readable recording medium other than the memory 810. The separate computer-readable recording medium may include computer-readable recording media such as a floppy drive, a disk, a tape, a DVD/CD-ROM drive, or a memory card. The memory 810 may store computer-executable instructions.

The processor 820 is an example of a computer configured to execute computer-executable instructions 821. The processor 820 may control the overall operation of the computer device. In addition, the processor 820 may control the computer device to perform the operation shown in the drawing.

The processor 820 according to an embodiment of the present disclosure may execute instructions for generating a plurality of major histocompatibility complex-T cell receptor (MHC-TCR) structures by using an amino acid sequence as an input to a first learning model, obtaining a plurality of first pMHC-TCR structures, comparing the plurality of first pMHC-TCR structures and the plurality of MHC-TCR structures and identifying a plurality of peptides corresponding to the plurality of first pMHC-TCR structures, generating a second pMHC-TCR structure for each of the plurality of peptides, on the basis of the plurality of MHC-TCR structures, calculating structural energy of the second pMHC-TCR structure, generating a data set on the basis of the structural energy and the plurality of peptides, and generating a second learning model that estimates whether the peptide will bind to the TCR on the basis of the data set.

The processor 820 according to an embodiment of the present disclosure may execute instructions for obtaining amino acid sequences of a plurality of peptides and MHC-TCR structures, generating pMHC-TCR structures based on the MHC-TCR structures for each of the plurality of peptides, calculating structural energy of the pMHC-TCR structure, and estimating whether at least one of a plurality of peptides will bind to a TCR using the structural energy as an input to the second learning model that estimates whether the peptide will bind to the TCR.

The processor 820 may be implemented as a digital signal processor (DSP) configured to process digital signals, a microprocessor, and a time controller (TCON). However, the processor 820 is not limited thereto, and may include one or more central processing unit (CPU), micro controller unit (MCU), micro processing unit (MPU), controller, application processor (AP), communication processor (CP), and ARM processor or defined by the terms. In addition, the processor 820 may be implemented as a system on chip (SoC) or large scale integration (LSI) with embedded processing algorithms or may be implemented in the form of a field programmable gate array (FPGA).

Meanwhile, the above-described operating method of the computer device may be implemented in the form of a computer-readable storage medium configured to store instructions or data executable by a computer or processor. It may be written as a program executable on a computer, and may be implemented on a general-purpose digital computer that operates such a computer-readable storage medium. The computer-readable storage medium may be read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tape, floppy disk, magneto-optical data storage device, optical data storage device, hard disk, and solid-state drive (SSD), and any device that may store instructions or software, related date, data files, and data structures and may provide instructions or software, related date, data files, and data structures to a processor or computer to allow the processor or computer to execute instructions.

The foregoing description of exemplary embodiments of the present disclosure is provided for purposes of illustration and explanation, and is not intended to limit the present disclosure to the precise forms disclosed. It will be understood that various modifications, substitutions, and changes may be made to the embodiments described herein without departing from the scope of the present disclosure. For example, although a series of operations has been described with reference to FIGS. 7 and 8, the order of the operations may be modified in other embodiments consistent with the principles of the present disclosure, and non-dependent operations may be performed concurrently.

Although the present disclosure has been described with reference to the preferred embodiments mentioned above, it will be understood that various modifications and variations can be made without departing from the spirit and scope of the present disclosure. Accordingly, such modifications and variations shall be included within the scope of the appended claims insofar as they fall within the spirit of the present disclosure.

1. A method for generating a learning model for estimating peptide-T cell receptor (TCR) binding, the method comprising:
generating a plurality of major histocompatibility complex-T cell receptor (MHC-TCR) structures by using an amino acid sequence as an input to a first learning model;
obtaining a plurality of first pMHC-TCR structures;
comparing the plurality of first pMHC-TCR structures and the plurality of MHC-TCR structures and identifying a plurality of peptides corresponding to the plurality of first pMHC-TCR structures;
generating a second pMHC-TCR structure for each of the plurality of peptides, on the basis of the plurality of MHC-TCR structures;
calculating structural energy of the second pMHC-TCR structure;
generating a data set on the basis of the structural energy and the plurality of peptides; and
generating a second learning model configured to estimate whether a peptide will bind to a TCR, on the basis of the data set.

2. The method of claim 1, wherein
the identifying of the plurality of peptides comprises
matching one first pMHC-TCR structure of the plurality of first pMHC-TCR structures with one MHC-TCR structure of the plurality of MHC-TCR structures, on the basis of structural similarity.

3. The method of claim 2, wherein the identifying of the plurality of peptides comprises
determining a peptide bound to the first pMHC-TCR as a first peptide that binds to a TCR, when MHC-TCR sequences are identical in the matched structure of the first pMHC-TCR structure and the MHC-TCR structure.

4. The method of claim 2, wherein
the identifying of the plurality of peptides comprises
determining a peptide bound to the first pMHC-TCR as a second peptide that does not bind to a TCR, when MHC sequences are identical in the matched structure of the first pMHC-TCR structure and the MHC-TCR structure.

5. The method of claim 2, wherein
the identifying of the plurality of peptides comprises
determining a peptide bound to the first pMHC-TCR as a third peptide that binds to the MHC but does not bind to a TCR, when the peptide bound to the first pMHC-TCR is estimated to bind to the MHC of the MHC-TCR structure in the matched structure of the first pMHC-TCR structure and the MHC-TCR structure.

6. The method of claim 1, wherein
the generating of the data set comprises
using the structural energy as a feature, and labeling the first peptide with a first value and the second peptide and the third peptide with a second value.

7. The method of claim 1, wherein
the generating of the second pMHC-TCR structure comprises:
generating a pMHC structure for each of the plurality of peptides, on the basis of a plurality of MHC structures respectively included in the plurality of MHC-TCR structures and STRUMP-I;
matching the pMHC structure with one of the plurality of MHC-TCR structures on the basis of a similar MHC; and
removing the MHC structure corresponding to the pMHC structure, and generating a pMHC-TCR structure on the basis of the MHC-TCR structure matched with a peptide corresponding to the pMHC structure.

8. The method of claim 1, wherein
the calculating of the structural energy comprises:
performing structural optimization of a backbone and a side chain of the second pMHC-TCR structure; and
calculating energy between the pMHC and the TCR, energy between the MHC and the TCR, and energy between the peptide and the TCR in the second pMHC-TCR structure.

9. The method of claim 1, wherein
the obtaining of the plurality of first pMHC-TCR structures comprises
obtaining the first pMHC-TCR structures from an RCSB_PDB database.

10. The method of claim 1, wherein
the amino acid sequence comprises
sequences of MHC, TCR-Alpha, and TCR-Beta connected by a linker formed of glutamic acid.

11. A method for estimating peptide-T cell receptor (TCR) binding, the method comprising:
obtaining MHC-TCR structures and amino acid sequences of a plurality of peptides;
generating a pMHC-TCR structure for each of the plurality of peptides, on the basis of the MHC-TCR structures;
calculating structural energy of the pMHC-TCR structure; and
estimating whether at least one of the plurality of peptides will bind to a TCR, by using the structural energy as an input to a second learning model configured to estimate whether a peptide will bind to a TCR.

12. A computer device for generating a learning model for estimating peptide-T cell receptor (TCR) binding comprising:
a memory including instructions; and
a processor configured to execute the instructions,
wherein the processor executes instructions for
generating a plurality of major histocompatibility complex-T cell receptor (MHC-TCR) structures by using an amino acid sequence as an input to a first learning model;
obtaining a plurality of first pMHC-TCR structures;
comparing the plurality of first pMHC-TCR structures and the plurality of MHC-TCR structures and identifying a plurality of peptides corresponding to the plurality of first pMHC-TCR structures;
generating a second pMHC-TCR structure for each of the plurality of peptides, on the basis of the plurality of MHC-TCR structures;
calculating structural energy of the second pMHC-TCR structure;
generating a data set on the basis of the structural energy and the plurality of peptides; and
generating a second learning model configured to estimate whether a peptide will bind to a TCR, on the basis of the data set.

13. A computer device for estimating peptide-T cell receptor (TCR) binding, the computer device comprising:
a memory comprising instructions; and
a processor configured to execute the instructions,
wherein the processor executes instructions for
obtaining MHC-TCR structures and amino acid sequences of a plurality of peptides;
generating a pMHC-TCR structure for each of the plurality of peptides, on the basis of the MHC-TCR structures;
calculating structural energy of the pMHC-TCR structure; and estimating whether at least one of the plurality of peptides will bind to a TCR, by using the structural energy as an input to a second learning model configured to estimate whether a peptide will bind to a TCR.
